(19) United States
(12) Patent Application Publication
(10) Pub. No.: US 2023/0414380 A1
(43) Pub. Date: Dec. 28, 2023

(54) METHOD AND APPARATUS FOR ENHANCING OPERATION OF LEG PROSTHESIS

(71) Applicant: UNIVERSITY OF CENTRAL FLORIDA RESEARCH FOUNDATION, INC., Orlando, FL (US)

(72) Inventor: Hwan CHOI, Orlando, FL (US)

(21) Appl. No.: 18/021,895

(22) PCT Filed: Aug. 18, 2021
(86) PCT No.: PCT/US2021/046565

§ 371 Date: Feb. 17, 2023

Related U.S. Application Data
(60) Provisional application No. 63/066,951, filed on Aug. 18, 2020.

(51) Int. Cl. A61F 2/66

(52) U.S. Cl. CPC A61F 2/6607; A61F 2002/607

(57) ABSTRACT

A method and apparatus for enhancing the operation of leg prosthesis is provided. The apparatus includes a core configured to be attached between a first portion and a second portion of the leg prosthesis. The first portion is configured to move relative to the second portion in a first plane. The core is configured to be moved from a first position to a second position relative to the leg prosthesis such that a stiffness of the core in the first plane is varied from a first stiffness to a second stiffness. The method includes the step of attaching the core between the first portion and the second portion of the leg prosthesis. The method further includes moving the first portion relative to the second portion in a first plane. The method further includes moving the core with a motor from a first position to a second position relative to the leg prosthesis such that a stiffness of the core in the first plane varies from a first stiffness to a second stiffness.

### BACKGROUND

Over 1.6 million people in the United States are living with lower limb amputation. This number is rising and is expected to double by 2050. Transtibial amputation, or below-knee amputation, has multiple disadvantages due to the loss of most of the calf muscle. Calf muscles, particularly the gastrocnemius and soles muscles, play an important role in supporting the body and propelling it forward.

### SUMMARY

Passive ankle foot prostheses are widely used due to their low cost, durability, and light weight. The main feature of these devices is their ability to recycle energy during walking. For example, from heel contact to midstance, the carbon fiber foot deforms from the body weight. As the center of mass of the body moves forward in the transition from midstance to terminal stance, the foot starts to return to its original shape, providing support and propulsion energy that was stored in the deformation of the foot. To provide high energy return, the feet of these devices are commonly made of carbon fiber, due to their high stiffness and their ability to largely elastically deform. Although there are many different designs of carbon fiber, passive ankle foot prostheses available, they are commonly comprised of a single or multiple layers of carbon fiber blades. Tuning the level of ankle stiffness is one key element for prescribing prostheses to people with lower limb amputation. There are many different walking conditions, i.e., incline, decline, soft, and rigid surfaces as well as different walking speeds experienced in daily living. Each different walking condition requires a different ankle stiffness to enable efficient walking.

However, the inventors of the present invention recognized that current standard of care passive prostheses only provides a single stiffness setting, leading to improper walking behaviors, i.e., walking asymmetry, increased musculature demands, and excessive joint load in different walking conditions. Thus, the inventors of the present invention recognized that it is crucial to have ankle prosthesis which can rapidly alter its ankle stiffness to provide efficient and comfortable walking for people with lower limb amputation.

Recently, a three-point bending and a pretension spring mechanism [1,2] were proposed to adjust prosthetic ankle stiffness. However, the three-point bending mechanism requires a metal frame which covers the entire foot to integrate a fulcrum, motor, and carbon fiber bar, resulting in a rigid and heavy device. The inventors of the present invention recognized that this rigid and heavy profile hinders the natural rolling motion during terminal stance and increases the metabolic cost required to carry the heavy prosthetic foot. Moreover, the inventors of the present invention recognized that the motor is placed on the distal end of the foot and may be vulnerable to impacts during dynamic tasks. The pretension mechanism requires a large mechanical spring and powerful actuator to compress the spring, its overall size is bulky, and the center of mass of the device is not aligned with the biological limb. Both designs also have a slow response in changing their stiffness. The three-point bending mechanism uses an acme screw to alter the position of the fulcrum of the ankle bending. The pretension mechanism uses a motor to compress the spring. These indirect ways to change stiffness are limited and cannot provide rapid stiffness changes leading to improper adaptations to new walking conditions.

To overcome the noted drawbacks of conventional prosthesis, the inventors of the present invention proposes a real-time adjustable stiffness ankle foot prosthesis that will enable a highly efficient energy recycling mechanism by mimicking the mechanism of the saddle spring found in mantis shrimp. The inventors developed an innovative bio-inspired shaped elastomer which enables the prosthesis to be light-weight and allow for changes in stiffness in a prompt manner.

In one embodiment, an apparatus is provided for enhancing operation of a leg prosthesis. The apparatus includes a core configured to be attached between a first portion and a second portion of the leg prosthesis, where the first portion is configured to move relative to the second portion in a first plane. The core is configured to be moved from a first position to a second position relative to the leg prosthesis such that a stiffness of the core in the first plane is varied from a first stiffness to a second stiffness.

In another embodiment, a method is provided for enhancing operation of a leg prosthesis. The method includes the step of attaching a core between a first portion and a second portion of the leg prosthesis. The method further includes moving the first portion relative to the second portion in a first plane. The method further includes moving the core with a motor from a first position to a second position relative to the leg prosthesis such that a stiffness of the core in the first plane varies from a first stiffness to a second stiffness.

In another embodiment, a leg prosthesis is provided with an apparatus according to the above embodiment mounted thereon.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is an image that illustrates an example of a mantis shrimp;

FIG. 1B is an image that illustrates an example of the mantis shrimp;

FIGS. 1C and 1D are images that illustrate an example of a saddle spring of the mantis shrimp, according to an embodiment;

FIGS. 1E through 1G are images that illustrate applied forces and resulting displacements of the saddle spring of FIGS. 1C and 1D;

FIG. 2A is an image that illustrates an example of a system for enhancing an operation of a leg prosthesis, according to an embodiment;

FIG. 2B is an image that illustrates an example of the saddle spring of the mantis shrimp modeled in a core of the system of FIG. 2A, according to an embodiment;

FIG. 2C is an image that illustrates an example of the core of the system of FIG. 2A, according to an embodiment;

FIG. 2D is an image that illustrates an example of cross-sectional view taken along the line 2D-2D in FIG. 2C, according to an embodiment;

FIG. 2E is an image that illustrates an example of the core, the hinge and the motor of the system of FIG. 2A, according to an embodiment;

FIG. 2F is an image that illustrates an example of a sensor and a controller mounted to the hinge of FIG. 2E, according to an embodiment;

FIG. 2G is a block diagram that illustrates the components of the system of FIG. 2A, according to an embodiment;

FIG. 3 is a flow chart that illustrates an example method for enhancing an operation of a leg prosthesis of FIG. 2A, according to an embodiment;

FIG. 4 is a block diagram that illustrates a computer system upon which an embodiment of the invention may be implemented; and

FIG. 5 illustrates a chip set upon which an embodiment of the invention may be implemented.

### DETAILED DESCRIPTION

A method and apparatus are described for enhancing the operation of leg prostheses and/or ankle prostheses. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of enhancing the operation and functionality of leg prostheses and/or ankle prostheses. For purposes of this invention, “leg prostheses” means one or more artificial body parts to replace any part of the leg and/or foot of a subject (e.g. human or non-human) that is not present (e.g. amputated). In an example embodiment, the leg prostheses is one or more artificial body parts that replace one or more portions of the leg below the knee (e.g. for a transtibial amputation). In still other embodiments, the leg prostheses is one or more artificial body parts that replace one or more portions of the leg above the knee (e.g. for subjects with above knee amputation). In other embodiments, the invention is described below in the context of improving the timing of stiffness adjustment of the leg prosthesis based on conditions of movement (e.g. speed of movement, incline of movement, surface of movement, etc.) of the user of the leg prosthesis. In still other embodiments, the invention is described below in the context of core design that can be applied to exoskeletal devices (e.g. ankle foot orthosis, knee brace, etc.).

### 1. Overview

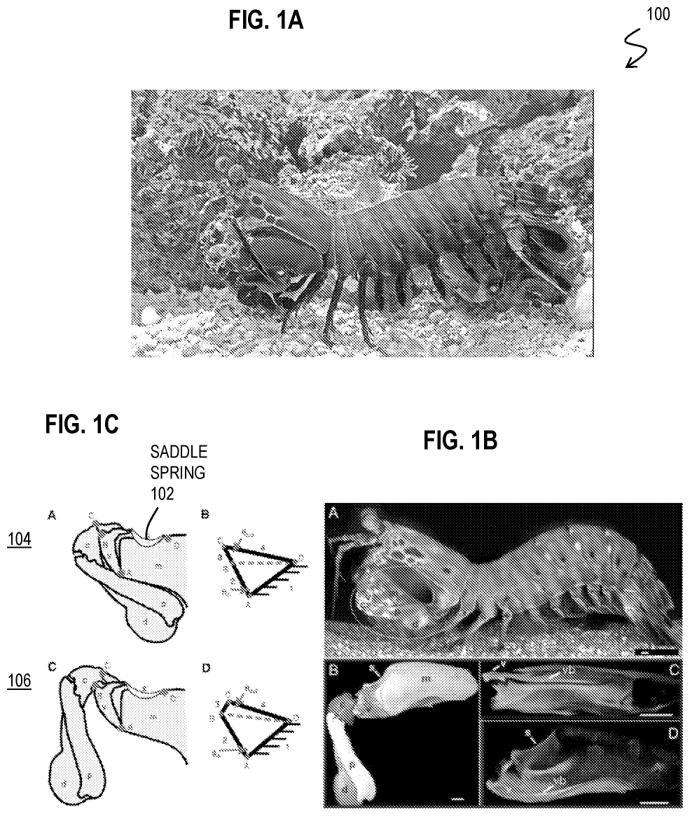

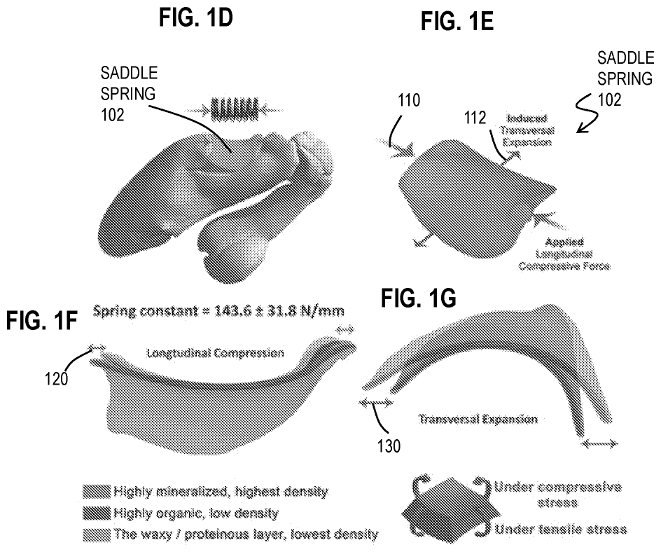

FIGS. 1A and 1B are images that illustrate an example of a mantis shrimp 100 [5], [6]. FIGS. 1C and 1D are images that illustrate an example of a saddle spring 102 of the mantis shrimp. During movement of a limb of the mantis shrimp 100 from a first position 104 to a second position 106 (FIG. 1C), a longitudinal force is applied to the saddle spring 102 (e.g. in the plane of FIG. 1C) and as a result the saddle spring 102 transversely expands (e.g. perpendicular to the plane of FIG. 1C).

FIGS. 1E through 1G are images that illustrate applied forces and resulting displacements of the saddle spring 102 of FIGS. 1C and 1D [5], [6]. As shown in FIG. 1E, a compression force is applied in a longitudinal direction 110 to the saddle spring 102 and consequently the saddle spring 102 transversely expands in a transverse direction 112 that is about orthogonal to the longitudinal direction 110. FIG. 1F shows a cross-section of the saddle spring 102 along the longitudinal direction 110 and depicts a longitudinal compression 120 based on the applied force in the longitudinal direction 110. FIG. 1G shows a cross-section of the saddle spring 102 along the transverse direction 112 and depicts a transverse expansion 130 based on the applied force in the longitudinal direction 110. The inventors recognized that the saddle spring 102 acts as a spring since upon releasing the applied force in the longitudinal direction 110, the saddle spring 102 would expand in the longitudinal direction 110 (e.g. to undo the longitudinal compression 120) and undo the transverse expansion 130, to return to its original shape prior to the application of the force in the longitudinal direction 110. In an example embodiment, the saddle spring 102 has a spring constant of about 143.6±31.8 Newton per millimeter (N/mm). It is has been recognized that the saddle spring 102 has particular characteristics (e.g. thin outer hard shell and thick inner relatively soft material) which could be implemented in a core used for a leg prosthesis. In an embodiment, the proposed design of the apparatus for enhancing the operation of leg prosthesis is inspired by the unique mechanism found in the saddle spring 102, which stores a large amount of energy with small deformation to catch its prey [4].

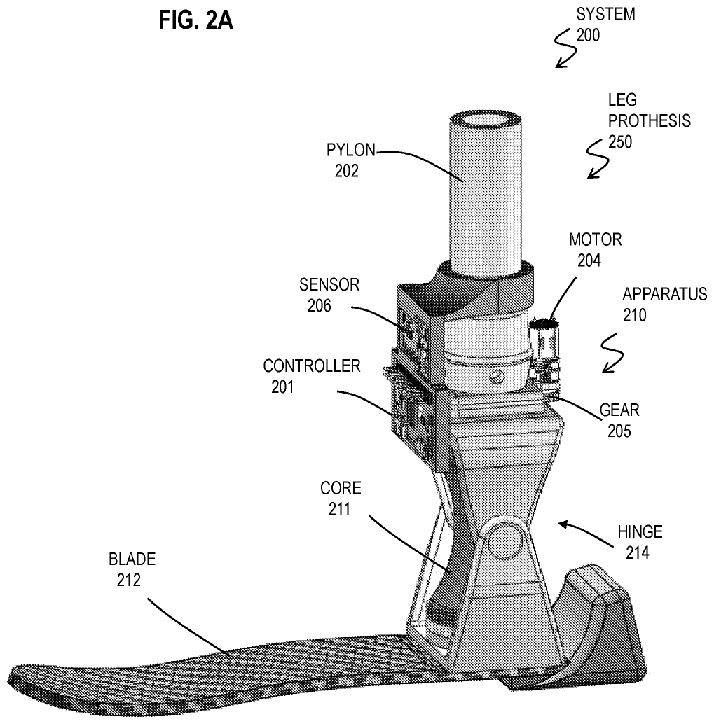

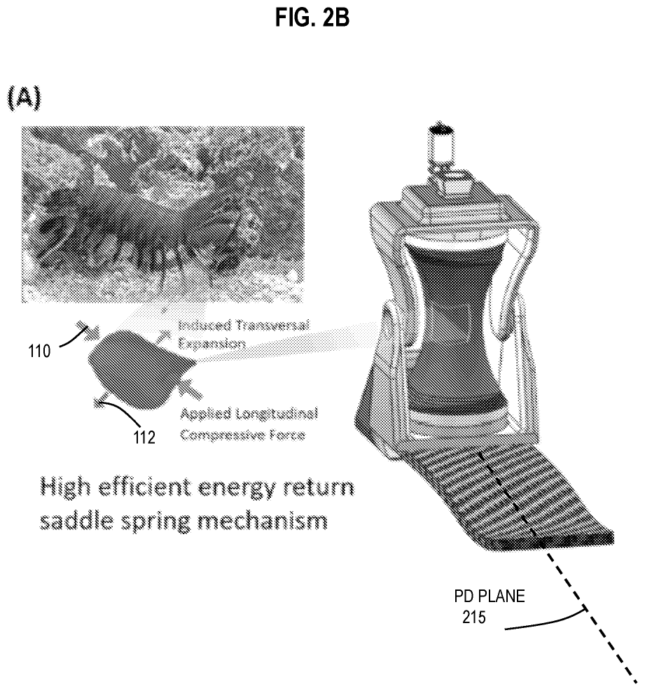

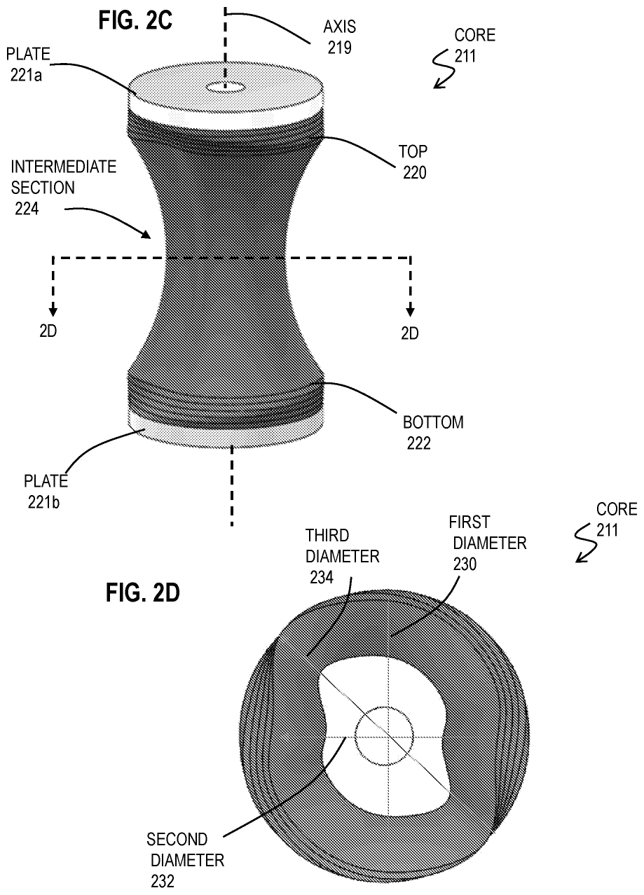

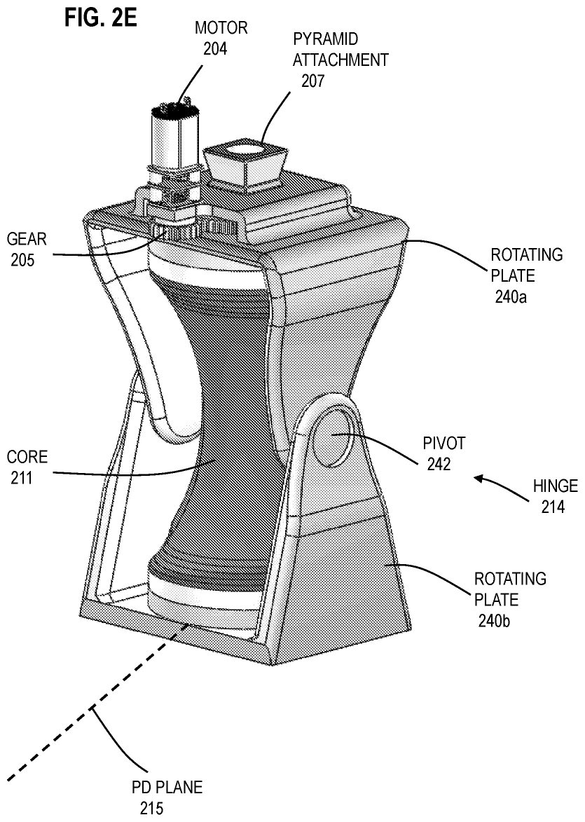

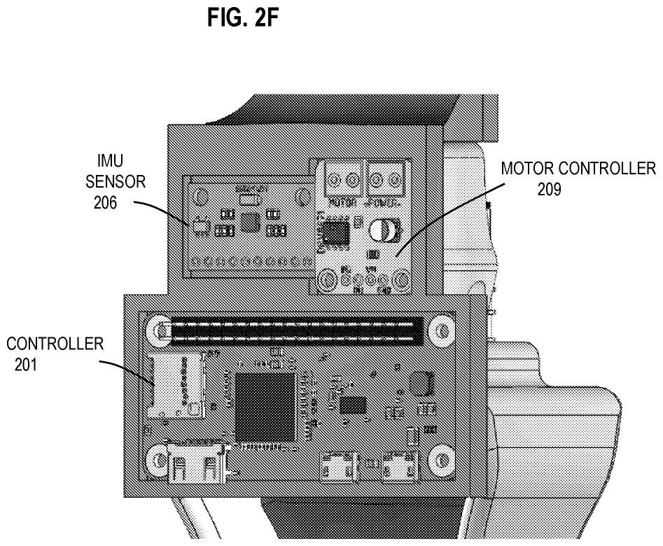

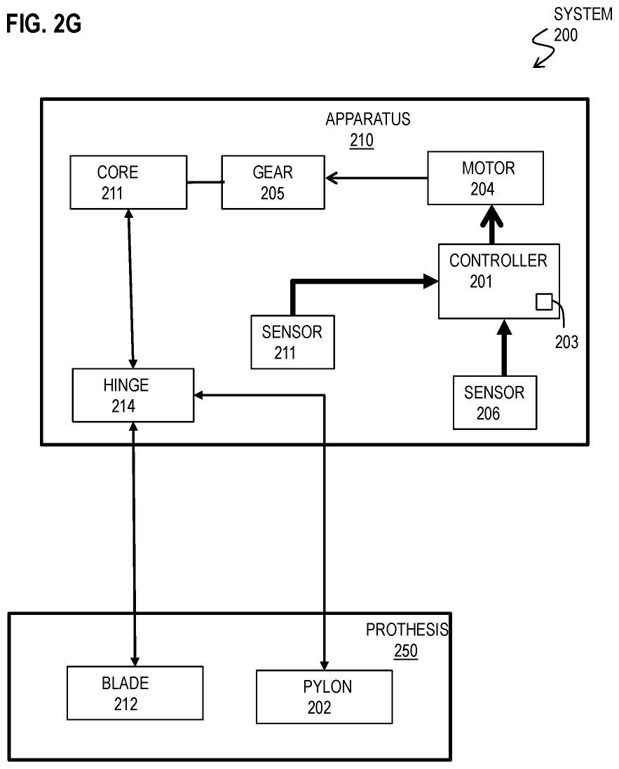

FIG. 2A is an image that illustrates an example of a system 200 for enhancing an operation of a leg prosthesis 250, according to an embodiment. In one embodiment, the leg prosthesis 250 includes a first portion (e.g. semi-rigid blade 212) and a second portion (e.g. pylon 202). The blade may take one of multiple forms, but generally relates to an elongated planar structure that has some flexibility and may include a curve. A pylon may include an elongated structure that is typically, though not necessarily cylindrical, and in specific embodiments has a channel or hollow chamber defined therein. Blades and pylons are terms known in the art, see U.S. Pat. Nos 7,288, 117; 9,687,365; 5,571,207; and U.S. Pat Pub 20020138153. In an example embodiment, the pylon 202 is secured to the leg of a user (e.g. at an amputation site). In one embodiment, the system 200 includes a hinge 214 that pivotally couples the semi-rigid blade 212 to the pylon 202 such that the semi-rigid blade 212 and the pylon 202 can rotate with respect to each other in a first plane. In an example embodiment, the hinge 214 is configured to rotate in the first plane (e.g. plantar-dorsiflexion plane or PD plane 215, see FIGS. 2B and 2E) such that the semi-rigid blade 212 and pylon 202 can rotate with respect to each other in the first plane. In one example embodiment, the hinge 214 is configured to rotate only in the first plane such that the semi-rigid blade 212 and pylon 202 can rotate with respect to each other only in the first plane. In another example embodiment, the semi-rigid blade 212 and pylon 202 are configured to rotate with respect to each other in more than one plane (e.g. PD plane 215 and a second plane orthogonal to the PD plane 215). As appreciated by one of ordinary skill in the art, during operation of the leg prosthesis 250, the pylon 202 and semi-rigid blade 212 rotate with respect to each other in the first plane (e.g. PD plane 215) based on a combination of effort of the user and ground reaction forces during the gait phases of the user.

In an embodiment, an apparatus 210 is provided to enhance the operation of the leg prosthesis 250. In one embodiment, the apparatus 210 excludes the leg prosthesis 250. In an example embodiment, the apparatus 210 is a kit that can be installed on an existing leg prosthesis to enhance the operation of an existing leg prosthesis (e.g. provide adjustable stiffness to the leg prosthesis based on movement conditions). In another example embodiment, the system 200 includes the apparatus 210 and the leg prosthesis 250.

In an embodiment, the apparatus 210 includes a core 211 configured to be attached between the first portion (e.g. semi-rigid blade 212) and the second portion (e.g. pylon 202) of the leg prosthesis 250. As appreciated by one of ordinary skill in the art, different conditions of movement of the leg prosthesis 250 (e.g. different speed, different incline, different surface, etc.) require different stiffness levels of the leg prosthesis 250. In an example embodiment, a running condition requires a greater stiffness level in the leg prosthesis 250 relative to a walking condition.

In an embodiment, the stiffness level of the leg prosthesis 250 can be adjusted by moving the core 211 from a first position to a second position (e.g. relative to the leg prosthesis 250). This advantageously permits the stiffness level of the leg prosthesis 250 to be adjusted, by moving the core 211 from the first position to the second position. In an embodiment, the apparatus 210 includes a sensor 206 to measure a value of a parameter that indicates the condition of movement (e.g. speed, incline, surface, etc.) of the leg prosthesis 250. In one embodiment, the sensor 206 transmits a signal to a controller 201 with the value of the parameter that indicates the condition of movement. In an embodiment, upon receiving the signal from the sensor 206, the controller 201 determines a desired level of stiffness for the leg prosthesis 250 and/or a position of the core 211 to achieve the desired level of stiffness, based on the received value of the parameter received from the sensor 206. In an example embodiment, the controller 201 transmits a signal to one or more components (e.g. motor 204, gear 205) to move the core 211 from a first position to a second position, such that the leg prosthesis 250 has the desired level of stiffness when the core 211 is moved to the second position.

In other embodiments, the position of the core 211 is manually adjusted (e.g. using a user input device 412, such as a smartphone) so that the user can manually adjust the level of stiffness of the leg prosthesis 250 (e.g. prior to going for a run, the user can manually adjust the position of the core 211 and thus manually adjust the level of stiffness of the leg prosthesis 250 to a desired level of stiffness for running). In this example embodiment, the user input device 412 is communicatively coupled (e.g. via a Bluetooth® connection) with the controller 201 and upon receiving a signal from the user input device 412 indicating the desired level of stiffness and/or a condition of movement, the controller 201 determines a desired position of the core 211 (to achieve the desired level of stiffness) and transmits a signal to the components (e.g. motor 204, gear 205, etc.) to move the core 211 from the first position to the desired position such that the desired level of stiffness is achieved.

FIG. 2B is an image that illustrates an example of the saddle spring 102 of the mantis shrimp 100 modeled in the core 211 of the system 200 of FIG. 2A, according to an embodiment [5], [6]. In an embodiment, the core 211 is made from a plurality of layers, where each layer is modeled based on the saddle spring 102 (e.g. where the longitudinal direction 110 is along a central longitudinal axis 219 of the core 211, see FIG. 2C). Thus, in an example embodiment, as the user moves with the leg prosthesis 250, the hinge 214 rotates in the first plane (e.g. PD plane 215) which compresses or expands the core 211 along the central longitudinal axis 219 of the core 211. Based on this compression or expansion of the core 211 along the central longitudinal axis 219, the core 211 respectively stores or releases energy along the central longitudinal axis 219 (e.g. in a similar manner that the saddle spring 202 stores or releases energy along the longitudinal direction 110). In an example embodiment, the hinge 214 and leg prosthesis 250 are configured to only rotate in the PD plane 215. Thus, in one embodiment, the unique mechanism of the saddle spring 102 is modeled into the design for the core 211 (e.g. an elastomer) which when fit onto the ankle foot prosthesis 250 can provide highly efficient energy return with a small amount of ankle dorsiflexion during walking. The core 211 is shaped such that it can store large amounts of force based on small deformation (e.g. along the axis 219).

FIG. 2C is an image that illustrates an example of the core 211 of the system 200 of FIG. 2A, according to an embodiment. FIG. 2D is an image that illustrates an example of cross-sectional view taken along the line 2D-2D in FIG. 2C, according to an embodiment. In an embodiment, the core 211 is configured to be moved (e.g. with the motor 204) from a first position to a second position relative to the leg prosthesis 250 such that a stiffness of the core 211 in the first plane is varied from a first stiffness to a second stiffness (e.g. desired level of stiffness). In some embodiments, the core 211 is configured to rotate about the central axis 219 of the core 211 from a first orientation to a second orientation (e.g. relative to the leg prosthesis 250 and/or PD plane 215) such that the stiffness of the core in the first plane is varied from the first stiffness to the second stiffness.

As shown in FIGS. 2C and 2D, an outer surface of the core 211 defines a variation in curvature from a top 220 to a bottom 222 of the core 211. In an embodiment, FIG. 2D is a cross-section of the core 211 through an intermediate section 224 (e.g. between the top 220 and bottom 222 of the core 211). In one embodiment, the core 211 is shaped similar to an hourglass (i.e. having a body with top and bottom sections and a middle section, wherein a middle section has a smaller circumference compared to the circumference of the top and bottom sections). However, the core 211 is not limited to taking any particular shape, provided that the core 211 provides different level of stiffness based rotation of the orientation of the core 211. Upper and lower plates 221a, 221b are provided where the upper and lower plates 221a, 221b are respectively secured to an inner surface of the upper and lower rotating plates 240a, 240b of the hinge 214 (FIG. 2E).

In one example embodiment, the intermediate section 224 is a section of the core 211 along the central axis 219 with a minimum cross-sectional area. As shown in FIG. 2D, a diameter of the core 211 at the intermediate section 224 varies, depending on an angle or orientation of the core 211. In an example embodiment, a first diameter 230 of the intermediate section 224 is larger than a second diameter 232 of the intermediate section 224. The variation in curvature of the core 211 from the top 220 to the bottom 222 is larger along a plane or orientation along the second diameter 232 (e.g. larger variation in the diameter of the core 211 from the top 220 to the bottom 222) than along a plane or orientation along the first diameter 230 (e.g. smaller variation in the diameter of the core 211 from the top 220 to the bottom 222). In an example embodiment, the stiffness of the core 211 along the first plane is based on the variation of curvature of the core 211 (from the top 220 to the bottom 222) along the first plane. Thus, in one example embodiment, rotating the core 211 such that the first diameter 230 is oriented along the first plane provides greater stiffness (e.g. due to lower variation in curvature along the first diameter 230) and rotating the core 211 such that the second diameter 232 is oriented along the first plane provides lower stiffness (e.g. due to higher variation in curvature along the second diameter 232).

As shown in FIG. 2D, a third diameter 234 is also provided with a distinct variation in curvature from the top 220 to the bottom 222 of the core 211. Thus, orienting the core 211 such that the third diameter 234 is aligned with the first plane provides a distinct level of stiffness of the core 211 in the first plane than the first and second diameters 230, 232 being aligned with the first plane. Although three diameters 230, 232, 234 of the core 211 are depicted, there is no limit to the number of distinct diameters that can be utilized, where each diameter provides a distinct variation in curvature and thus distinct level of stiffness when the core 211 is rotated. Additionally, although FIG. 2D depicts that distinct diameters of the intermediate section 224 can be used to provide different variations in curvature and thus different stiffness levels along the first plane, in other embodiments, a thickness of an outer shell of the core 211 is varied from the top 220 to the bottom 222 at different angles around the circumference of the core 211. In these embodiments, the core 211 can provide different levels of stiffness along the first plane based on rotation of the core 211 so that different variations in thickness of the core 211 are aligned with the first plane. In one example embodiment, the core 211 could be cylindrically shaped (e.g. with a fixed outer diameter from the top 220 to the bottom 222) but with different stiffness levels at different orientations based on the thickness of the outer shell having different variations (e.g. from the top 220 to the bottom 222) at different orientations of the core 211.

It is recognized herein that since the curvature of the saddle spring 102 determines its level of stiffness, aspects of the geometry of the mantis shrimp structure could be implemented into a core 211 shape (e.g. an hourglass-like shape) that allows for continuous changes in curvature as the orientation of the elastomer varies. The saddle spring 102 of mantis shrimp has a rigid outer layer and compliant inner layer, which permits efficient energy return. In an example embodiment, to simulate the structure and function of the mantis shrimp saddle spring 102, two different carbon fiber sheets (e.g. a horizontal layer and vertical layer of carbon fibers) were used. In one example embodiment, the horizontal layer of carbon fiber has a stiff response, while the vertical layer of carbon fiber has a compliant response when the force is applied vertically. In this example embodiment, the horizontal layer of carbon fiber is placed on the outside of the core 211 (e.g. to simulate the stiff outer shell of the saddle spring 102) and a vertical layer of carbon fiber on inside of the core 211 (e.g. to simulate the compliant inner layer of the saddle spring 102). It was recognized that this continuous curvature would allow for different stiffness as the entire core 211 (e.g. elastomer) rotates, e.g. from the control of a direct current brushless motor 204 installed on the posterior of the prosthesis 250. In an example embodiment, the design of the core 211 will change the level of stiffness in a prompt manner since the motor 204 directly rotates the core 211. In an example embodiment, in order to store energy during stance phase, a rigid material is utilized for the foot blade 212.

FIG. 2E is an image that illustrates an example of the core 211, the hinge 214 and the motor 204 of the system 200 of FIG. 2A, according to an embodiment. In an embodiment, the hinge 214 includes a first section (e.g. top rotating plate 240a) attached to the pylon 202 of the leg prosthesis 250 and a second section (e.g. bottom rotating plate 240b) attached to the blade 212 of the leg prosthesis 250. In an example embodiment, the top rotating plate 240a features a pyramid attachment 207 that is configured to attach the top rotating plate 240a to the pylon 202. In an embodiment, the upper plate 240a and lower plate 240b are configured to rotate (e.g. about pivot 242) in the first plane (e.g. PD plane 215). In one example embodiment, the hinge 240 is configured to rotate in only the PD plane 215.

As shown in FIG. 2E, in one embodiment the core 211 is mounted between the upper rotating plate 240a and the lower rotating plate 240b of the hinge 240. In an example embodiment, an upper plate 221a of the core 211 (FIG. 2C) is secured to an inside surface of the upper rotating plate 240a and a lower plate 221b of the core 211 is secured to an inside surface of the lower rotating plate 240b. In one embodiment, the core 211 is secured to the hinge 240 such that rotation of the upper rotating plate 240a relative to the lower rotating plate 240b in the PD plane 215 causes compression or expansion of the core 211 in the PD plane 215. In an example embodiment, a top of the core 211 (e.g. top 220 and/or the upper plate 211a) and a bottom of the core 211 (e.g. bottom 222 and/or the lower plate 211b) are circular to allow an equal bending moment regardless of the orientation of the core 211 (e.g. within the first plane).

As further shown in FIG. 2E, in one embodiment, the apparatus 210 includes a gear 205 operatively coupled to the motor 204 such that the motor 204 is configured to rotate the gear 205 which in turn causes the core 211 to rotate from a first orientation (e.g. first diameter 230 aligned in the PD plane 215) to a second orientation (e.g. second diameter 232 aligned in the PD plane 215) based on a signal received at the motor 204 from the controller 201. In one example embodiment, one or more of the sensor 206, the motor 204, the gear 205 and the controller 201 are mounted to the upper rotating plate 240a of the hinge 214. FIG. 2F is an image that illustrates an example of the sensor 206 (e.g. IMU sensor) and the controller 201 mounted to the hinge 214 of FIG. 2E, according to an embodiment. Additionally, in one embodiment, FIG. 2F depicts a motor controller 209 that is communicatively coupled to the controller 201 and the motor 204. In this example embodiment, the controller 201 transmits a signal to the motor controller 209 and the motor controller 209 subsequently transmits a signal to the motor 204 to initiate the movement (e.g. rotation) of the core 211.

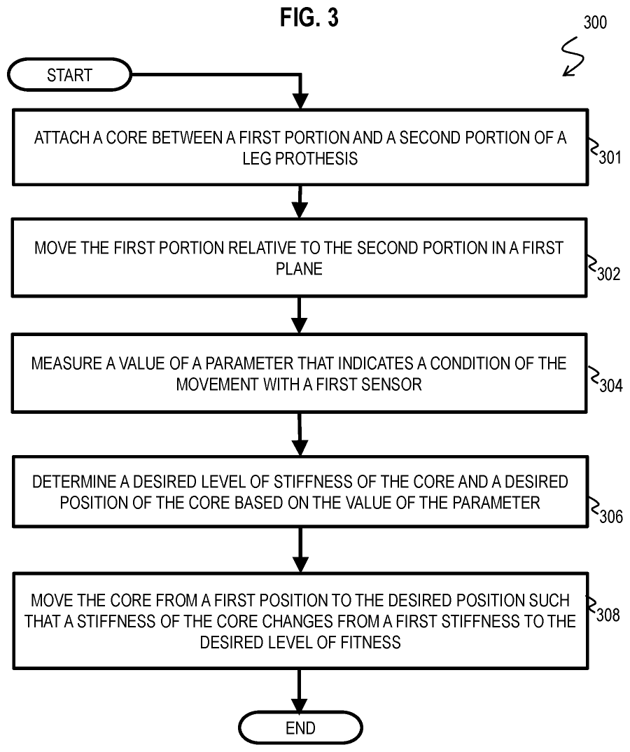

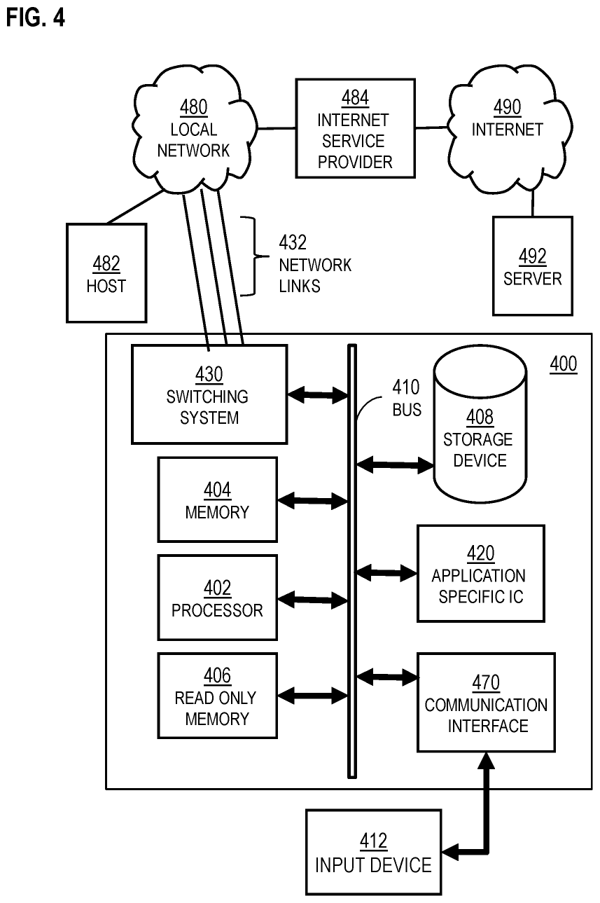

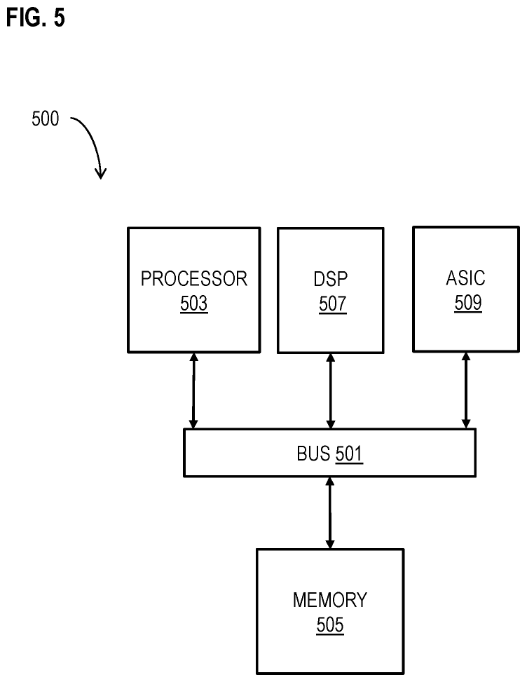

FIG. 2G is a block diagram that illustrates the components of the system 200 of FIG. 2A, according to an embodiment. Thin lines (1.5 point) in FIG. 2G indicate mechanical coupling between components of the system and thick lines (3.5 point) indicate communicative coupling between the components of the system. In an embodiment, the apparatus 210 of the system 200 includes the controller 201, such as a computer system described below with reference to FIG. 4, or a chip set described below with reference to FIG. 5. A memory 203 of the controller 201 includes instructions to perform one or more steps of the method 300 based on the flowchart of FIG. 3.

In an embodiment, the apparatus 210 includes a first sensor 206 configured to measure a value of a parameter that indicates a condition of movement (e.g. one or more of a speed, an incline, a surface of movement, etc.) of a user wearing the legal prosthesis 250. In an example embodiment, the first sensor 206 is an inertial measurement unit (IMU). In an embodiment, the motor 204 is configured to move the core 211 (e.g. rotate the core 211) from the first position to the second position (e.g. from a first orientation to a second orientation). In an example embodiment, the motor 204 is configured to displace the gear 205 which in turn rotates the core 211 (e.g. about the central axis 219).

In an embodiment, the controller 201 is communicatively coupled to the first sensor 206 and the motor 204. During operation of the system, the first sensor 206 measures the value of the parameter (e.g. value of an acceleration measured by the IMU sensor due ground forces enacted on the leg prosthesis 150 at one or more time increments) and transmits a first signal indicating the value of the parameter to the controller 201. In an example embodiment, the first sensor 206 measures the value of the parameter that indicates one or more of a speed, an incline angle and a surface of movement of the user wearing the leg prosthesis 250.

In one embodiment, the controller 201 receives the first signal from the first sensor 206 indicating the value of the parameter. The controller 201 determines a desired level of stiffness based on the received value of the parameter from the first sensor 206 and/or further determines a desired position (e.g. desired orientation) of the core 211 to achieve the desired level of stiffness. In an example embodiment, the memory 203 of the controller 201 stores first data that indicates a desired level of stiffness of the core 211 in the first plane based on the value of the parameter and/or second data that indicates a desired position (e.g. desired orientation) of the core 211 in the first plane to achieve the desired level of stiffness. In one embodiment, upon determining a desired position (e.g. desired orientation) of the core 211, the controller 201 transmits a second signal to the motor 204 to cause the motor 204 to move the core 211 from the first position to the desired position such that the desired level of stiffness is achieved. In one example embodiment, upon determining the desired position (e.g. desired orientation) of the core 211, the controller 201 transmits the second signal to the motor controller 209 (FIG. 2F) which in turn transmits a signal to the motor 204 to cause the motor 204 to move from the first position to the desired position.

In an example embodiment, upon the controller 201 receiving the first signal from the first sensor 206 indicating that the speed of movement of the user increased from a first speed (e.g. walking speed) to a second speed (e.g. jogging or running speed), the controller 201 determines a desired level of stiffness (e.g. from data in the memory 203) based on the second speed and/or a desired position (e.g. desired orientation) of the core 211 to achieve the desired level of stiffness in the first plane. In an example embodiment, the controller 201 transmits the second signal to the motor 204 (e.g. or to the motor controller 209 which subsequently transmits a signal to the motor 204) to cause the core 211 to move from the first position to the desired position, where the desired level of stiffness of the core 211 in the desired position is greater than the first stiffness of the core 211 in the first position.

As shown in FIG. 2G, in some embodiments the apparatus 210 includes a second sensor 211 communicatively coupled with the motor 204 (e.g. or to the motor controller 209) and configured to determine that the core 211 has moved from the first position to the second position (e.g. desired position). In one embodiment, the second sensor 211 is an encoder attached to the gear 205. In an example embodiment, the second sensor 211 transmits a third signal to the controller 201 (or the motor controller 209) upon determining that the core 211 has moved from the first position to the second position (e.g. desired position). In an example embodiment, upon receiving the third signal from the second sensor 211, the controller 201 (or the motor controller 209) transmits a signal to the motor 204 to stop moving the core 211 (e.g. since the core 211 is in the desired position and thus providing the desired level of stiffness).

FIG. 3 is a flow chart that illustrates an example method 300 for enhancing the operation of a leg prosthesis. Although steps are depicted in FIG. 3 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 301, the core is attached between the first portion and the second portion of the leg prosthesis. In one embodiment, in step 301 the core 211 is attached between the blade 212 and the pylon 202 of the system 200. In an example embodiment, in step 301 the core 211 is mounted within the hinge 214 (e.g. upper plate 221a is mounted to the top rotating plate 240a and the lower plate 221b is mounted to the bottom rotating plate 240b) and the hinge 214 is attached to the leg prosthesis 250 (e.g. upper rotating plate 240a is secured to the pylon 202 and the lower rotating plate 240b is secured to the blade 212).

In step 302, the first portion of the leg prosthesis is moved relative to the second portion of the leg prosthesis in the first plane. In an embodiment, after attaching the leg prosthesis 250 to the user in step 301, in step 302 the user initiates a gait cycle with the leg prosthesis 250 along a surface. In an example embodiment, in step 302 the blade 212 moves within the PD plane 215 relative to the pylon 202 (e.g. due to effort of the user and/or ground reaction forces).

In step 304, a value of a parameter is measured that indicates a condition of movement of the leg prosthesis 250 in step 302. In one embodiment, in step 304 the value of the parameter is measured by the first sensor 206. In an example embodiment the parameter includes one or more of speed, incline, surface of movement, and any other parameter that can be used to characterize a movement of the leg prosthesis 250. In an example embodiment the first sensor 206 is an IMU sensor and/or is configured to measure the value of the parameter at incremental time periods. In an example embodiment, in step 304 the first sensor 206 transmits a first signal to the controller 201 that indicates the value of the parameter.

In step 306, a desired level of stiffness for the core is determined based on the value of the parameter measured in step 304. In one embodiment, in step 306 a desired position (e.g. desired orientation) of the core 211 is determined based on the desired level of stiffness and/or the value of the parameter. In an example embodiment, the memory 203 of the controller 201 stores first data that indicates the desired level of stiffness (e.g. based on the value of the parameter) and/or second data that indicates the desired position (e.g. based on the desired level of stiffness). In an embodiment, in step 306 the controller 201 receives the first signal from the first sensor 206 and uses the measured value of the parameter to determine the desired level of stiffness and/or desired position of the core 211 to achieve the desired level of stiffness.

In an example embodiment, the first data and the second data are obtained during a calibration process, e.g. where the leg prosthesis 250 is moved at different conditions of movement (e.g. different speeds, different inclines, etc.) and the level of stiffness of the core 211 is measured at different positions of the core 211. The position of the core 211 at which the desired level of stiffness is attained is stored in the memory 203 for each movement condition. In an example embodiment, the desired level of stiffness is known for different conditions of movement.

In step 308, the core is moved from a first position to a second position (e.g. desired position) such that the stiffness of the core in the second position is the desired level of stiffness determined in step 306. In an embodiment, in step 308 the controller 201 (or the motor controller 209) transmits a second signal to the motor 204 to cause the motor 204 (e.g. and gear 205) to move the core 211 from the first position to the desired position (e.g. or from the first orientation to the desired orientation).

In an example embodiment, in step 308 the second sensor 211 measures a position of the core 211 and transmits a third signal to the controller 201 (or the motor controller 209) indicating the position of the core 211 during step 308. In an example embodiment, upon the controller 201 (or motor controller 209) determining that the current position of the core 211 (e.g. from the third signal) corresponds to the desired position, the controller 201 (or motor controller 209) transmits a fourth signal to the motor 204 to stop movement of the core 211.

In an embodiment, the method 300 includes a loop which repeats steps 302 through 308. For each loop of steps 304 through 308, if the movement condition of the leg prosthesis (step 304) does not change, then no action is taken in steps 306 and 308.

In an embodiment, step 304 measures a change in the value of the parameter (e.g. between one or more consecutive time increments) and if the measured change is less than a threshold value, steps 306 and 308 are not performed. In this embodiment, if the measured change is greater than a threshold value, steps 306 and 308 are performed. Similarly, in this example embodiment, in step 306 a desired change in the level of stiffness is determined and a change in the position of the core (e.g. to achieve the desired change in the level of stiffness). In this example embodiment, step 308 involves moving the core based on the change in the position of the core determined in step 306.

### 2. Hardware Overview

FIG. 4 is a block diagram that illustrates a computer system 400 upon which an embodiment of the invention may be implemented. Computer system 400 includes a communication mechanism such as a bus 410 for passing information between other internal and external components of the computer system 400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 400, or a portion thereof, constitutes a means for performing one or more steps of one or more methods described herein.

A sequence of binary digits constitutes digital data that is used to represent a number or code for a character. A bus 410 includes many parallel conductors of information so that information is transferred quickly among devices coupled to the bus 410. One or more processors 402 for processing information are coupled with the bus 410. A processor 402 performs a set of operations on information. The set of operations include bringing information in from the bus 410 and placing information on the bus 410. The set of operations also typically include comparing two or more units of information, shifting positions of units of information, and combining two or more units of information, such as by addition or multiplication. A sequence of operations to be executed by the processor 402 constitutes computer instructions.

Computer system 400 also includes a memory 404 coupled to bus 410. The memory 404, such as a random access memory (RAM) or other dynamic storage device, stores information including computer instructions. Dynamic memory allows information stored therein to be changed by the computer system 400. RAM allows a unit of information stored at a location called a memory address to be stored and retrieved independently of information at neighboring addresses. The memory 404 is also used by the processor 402 to store temporary values during execution of computer instructions. The computer system 400 also includes a read only memory (ROM) 406 or other static storage device coupled to the bus 410 for storing static information, including instructions, that is not changed by the computer system 400. Also coupled to bus 410 is a non-volatile (persistent) storage device 408, such as a magnetic disk or optical disk, for storing information, including instructions, that persists even when the computer system 400 is turned off or otherwise loses power.

Information, including instructions, is provided to the bus 410 for use by the processor from an external input device 412, such as a keyboard containing alphanumeric keys operated by a human user, or a sensor. A sensor detects conditions in its vicinity and transforms those detections into signals compatible with the signals used to represent information in computer system 400. Other external devices coupled to bus 410, used primarily for interacting with humans, include a display device 414, such as a cathode ray tube (CRT) or a liquid crystal display (LCD), for presenting images, and a pointing device 416, such as a mouse or a trackball or cursor direction keys, for controlling a position of a small cursor image presented on the display 414 and issuing commands associated with graphical elements presented on the display 414.

In the illustrated embodiment, special purpose hardware, such as an application specific integrated circuit (IC) 420, is coupled to bus 410. The special purpose hardware is configured to perform operations not performed by processor 402 quickly enough for special purposes. Examples of application specific ICs include graphics accelerator cards for generating images for display 414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 400 also includes one or more instances of a communications interface 470 coupled to bus 410. Communication interface 470 provides a two-way communication coupling to a variety of external devices that operate with their own processors, such as printers, scanners and external disks. In general, the coupling is with a network link 478 that is connected to a local network 480 to which a variety of external devices with their own processors are connected. For example, communication interface 470 may be a parallel port or a serial port or a universal serial bus (USB) port on a personal computer. In some embodiments, communications interface 470 is an integrated services digital network (ISDN) card or a digital subscriber line (DSL) card or a telephone modem that provides an information communication connection to a corresponding type of telephone line. In some embodiments, a communication interface 470 is a cable modem that converts signals on bus 410 into signals for a communication connection over a coaxial cable or into optical signals for a communication connection over a fiber optic cable. As another example, communications interface 470 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 470 sends and receives electrical, acoustic or electromagnetic signals, including infrared and optical signals, that carry information streams, such as digital data.

The term computer-readable medium is used herein to refer to any medium that participates in providing information to processor 402, including instructions for execution. Such a medium may take many forms, including, but not limited to, non-volatile media, volatile media and transmission media. Non-volatile media include, for example, optical or magnetic disks, such as storage device 408. Volatile media include, for example, dynamic memory 404. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 402, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 402, except for carrier waves and other signals.

Logic encoded in one or more tangible media includes one or both of processor instructions on a computer-readable storage media and special purpose hardware, such as ASIC *420.

Network link 478 typically provides information communication through one or more networks to other devices that use or process the information. For example, network link 478 may provide a connection through local network 480 to a host computer 482 or to equipment 484 operated by an Internet Service Provider (ISP). ISP equipment 484 in turn provides data communication services through the public, world-wide packet-switching communication network of networks now commonly referred to as the Internet 490. A computer called a server 492 connected to the Internet provides a service in response to information received over the Internet. For example, server 492 provides information representing video data for presentation at display 414.

The invention is related to the use of computer system 400 for implementing the techniques described herein. According to one embodiment of the invention, those techniques are performed by computer system 400 in response to processor 402 executing one or more sequences of one or more instructions contained in memory 404. Such instructions, also called software and program code, may be read into memory 404 from another computer-readable medium such as storage device 408. Execution of the sequences of instructions contained in memory 404 causes processor 402 to perform the method steps described herein. In alternative embodiments, hardware, such as application specific integrated circuit 420, may be used in place of or in combination with software to implement the invention. Thus, embodiments of the invention are not limited to any specific combination of hardware and software.

The signals transmitted over network link 478 and other networks through communications interface 470, carry information to and from computer system 400. Computer system 400 can send and receive information, including program code, through the networks 480, 490 among others, through network link 478 and communications interface 470. In an example using the Internet 490, a server 492 transmits program code for a particular application, requested by a message sent from computer 400, through Internet 490, ISP equipment 484, local network 480 and communications interface 470. The received code may be executed by processor 402 as it is received or may be stored in storage device 408 or other non-volatile storage for later execution, or both. In this manner, computer system 400 may obtain application program code in the form of a signal on a carrier wave.

Various forms of computer readable media may be involved in carrying one or more sequence of instructions or data or both to processor 402 for execution. For example, instructions and data may initially be carried on a magnetic disk of a remote computer such as host 482. The remote computer loads the instructions and data into its dynamic memory and sends the instructions and data over a telephone line using a modem. A modem local to the computer system 400 receives the instructions and data on a telephone line and uses an infra-red transmitter to convert the instructions and data to a signal on an infra-red a carrier wave serving as the network link 478. An infrared detector serving as communications interface 470 receives the instructions and data carried in the infrared signal and places information representing the instructions and data onto bus 410. Bus 410 carries the information to memory 404 from which processor 402 retrieves and executes the instructions using some of the data sent with the instructions. The instructions and data received in memory 404 may optionally be stored on storage device 408, either before or after execution by the processor 402.

FIG. 5 illustrates a chip set 500 upon which an embodiment of the invention may be implemented. Chip set 500 is programmed to perform one or more steps of a method described herein and includes, for instance, the processor and memory components described with respect to FIG. 4 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 500, or a portion thereof, constitutes a means for performing one or more steps of a method described herein.

In one embodiment, the chip set 500 includes a communication mechanism such as a bus 501 for passing information among the components of the chip set 500. A processor 503 has connectivity to the bus 501 to execute instructions and process information stored in, for example, a memory 505. The processor 503 may include one or more processing cores with each core configured to perform independently. A multi-core processor enables multiprocessing within a single physical package. Examples of a multi-core processor include two, four, eight, or greater numbers of processing cores. Alternatively, or in addition, the processor 503 may include one or more microprocessors configured in tandem via the bus 501 to enable independent execution of instructions, pipelining, and multithreading. The processor 503 may also be accompanied with one or more specialized components to perform certain processing functions and tasks such as one or more digital signal processors (DSP) 507, or one or more application-specific integrated circuits (ASIC) 509. A DSP 507 typically is configured to process real-world signals (e.g., sound) in real time independently of the processor 503. Similarly, an ASIC 509 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 503 and accompanying components have connectivity to the memory 505 via the bus 501. The memory 505 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 505 also stores the data associated with or generated by the execution of one or more steps of the methods described herein.

### 3. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 4. References

[1] M. K. Shepherd and E. J. Rouse, “The VSPA Foot: A Quasi-Passive Ankle-Foot Prosthesis With Continuously Variable Stiffness,” in IEEE Transactions on Neural Systems and Rehabilitation Engineering, vol. 25, no. 12, pp. 2375-2386, December 2017, doi: 10.1109/SNSRE.2017.2750113.
[2] L. M. Mooney, C. H. Lai and E. J. Rouse, “Design and characterization of a biologically inspired quasi-passive prosthetic ankle-foot,” 2014 36th Annual International Conference of the IEEE Engineering in Medicine and Biology Society, Chicago, IL, 2014, pp. 1611-1617, doi: 10.1109/EMBC.2014.6943913.
[3] E. M. Glanzer and P. G. Adamczyk, “Design and Validation of a Semi-Active Variable Stiffness Foot Prosthesis,” in IEEE Transactions on Neural Systems and Rehabilitation Engineering, vol. 26, no. 12, pp. 2351-2359, December 2018, doi: 10.1109/TNSRE.2018.2877962.
[4] Tadayon, M., Amini, S., Wang, Z. and Miserez, A., 2018. Biomechanical design of the mantis shrimp saddle: a biomineralized spring used for rapid raptorial strikes. iScience, 8, pp. 271-282.
[5] https://onlinelibrary.wiley.com/doi/full/10.1002/adfm.201502987
[6] http://jeb.biologists.org/content/210/20/3677

What is claimed is:

1. An apparatus comprising:
a core configured to be attached between a first portion and a second portion of a leg prosthesis wherein the first portion is configured to move relative to the second portion in a first plane;
wherein the core is configured to be moved from a first position to a second position relative to the leg prosthesis such that a stiffness of the core in the first plane is varied from a first stiffness to a second stiffness.

2. The apparatus of claim 1, wherein the first portion is a blade and the second portion is a pylon and wherein the first plane is a plantar-dorsiflexion (PD) plane.

3. The apparatus of claim 1, wherein the core is configured to rotate about a central axis of the core from a first orientation to a second orientation relative to the leg prosthesis such that the stiffness of the core in the first plane is varied from the first stiffness to the second stiffness.

4. The apparatus of claim 3, wherein an outer surface of the core defines a variation in curvature from a top of the core to a bottom of the core, and wherein the first stiffness is based on a first variation of the curvature in the first orientation and wherein the second stiffness is based on a second variation of the curvature in the second orientation that is different than the first variation.

5. The apparatus of claim 4, wherein the top and the bottom of core define a first area and wherein an intermediate section of the core between the top and the boom defines a second area that is smaller than the first area.

6. The apparatus of claim 5, wherein the core has an hourglass shape.

7. The apparatus of claim 1, further comprising a hinge with a first section attached to the first portion of the leg prosthesis and a second section attached to the second portion of the leg prosthesis, wherein the first section is configured to rotate relative to the second section in the first plane.

8. The apparatus of claim 7, wherein the core is mounted between the first section and the second section of the hinge such that rotation of the first section relative to the second section in the first plane causes compression or expansion of the core in the first plane.

9. The apparatus of claim 7, wherein the first section and the second section are a pair of free rotating plates connected at a pivot axis such that the rotating plates are configured to rotate about the pivot axis in the first plane.

10. The apparatus of claim 1, further comprising:
a first sensor configured to measure a value of a parameter that indicates a condition of movement of a user wearing the legal prosthesis;
a motor configured to move the core from the first position to the second position; and
a controller communicatively coupled to the first sensor and the motor, wherein the controller is configured to receive a first signal from the first sensor indicating the value of the parameter and wherein the controller is configured to transmit a second signal to the motor to cause the core to move from the first position to the second position.

11. The apparatus of claim 10, wherein the first sensor is configured to measure the value of the parameter that indicates one or more of a speed, an incline angle and a surface of movement of the user wearing the leg prosthesis.

12. The apparatus of claim 11, wherein upon receiving the first signal from the first sensor indicating that the speed of movement of the user increased from a first speed to a second speed, the controller is configured to transmit the second signal to the motor to cause the core to move from the first position to the second position, wherein the second stiffness of the core in the second orientation is greater than the first stiffness of the core in the first orientation.

13. The apparatus of claim 10,
wherein the core is configured to rotate about a central axis of the core from a first orientation to a second orientation relative to the leg prosthesis such that the stiffness of the core in the first plane is varied from the first stiffness to the second stiffness; and (2C-2D)
wherein a gear operatively coupled to the motor is configured to rotate the core from the first orientation to the second orientation based on the second signal received from the controller.

14. The apparatus of claim 10, further comprising a second sensor communicatively coupled with the motor and configured to determine that the core has moved from the first position to the second position and wherein the second sensor is configured to transmit a third signal upon determining that the core has moved from the first position to the second position and wherein the motor is configured to stop moving the core upon receiving the third signal from the second sensor.

15. The apparatus of claim 10, further comprising a hinge with a first section attached to the first portion of the leg prosthesis and a second section attached to the second portion of the leg prosthesis such that the first section is configured to rotate relative to the second section in the first plane;
wherein the first sensor, the motor and the controller are mounted to the first section of the hinge.

16. The apparatus of claim 1, wherein the first plane is plantar-dorsiflexion plane.

17. A system comprising:
the apparatus of claim 1; and
the leg prosthesis including the first portion and the second portion.

18. The system of claim 17, wherein the first portion is configured to move relative to the second portion in only the first plane.

19. The system of claim 17, wherein the first portion is a blade and the second portion is a pylon of the leg prosthesis; and wherein the system further comprises a hinge including a first section attached to the blade and the second section attached to the pylon, wherein the first section is configured to rotate relative to the second section in only the first plane.

20. A method comprising:
attaching a core between a first portion and a second portion of a leg prosthesis;
moving, in a first plane, the first portion relative to the second portion;
moving, with a motor, the core from a first position to a second position relative to the leg prosthesis such that a stiffness of the core in the first plane varies from a first stiffness to a second stiffness.

21. The method of claim 20, further comprising:
measuring, with a first sensor, a value of a parameter that indicates a condition of movement of a user during the moving of the first portion relative to the second portion;
transmitting, from the first sensor, a first signal to a controller indicating the value of the parameter;
determining, with the controller, a desired level of stiffness for the core and a desired position of the core based on the value of the parameter, wherein the second position is the desired position and the second stiffness is the desired level of stiffness; and
transmitting, from the controller, a second signal to a motor to initiate the moving step from the first position to the desired position such that the stiffness of the core in the first plane varies from the first stiffness to the desired level of stiffness.

22. The method of claim 21, further comprising:
measuring, with a second sensor, the position of the core during the moving step from the first position to the desired position;
transmitting, from the second sensor, a second signal to the controller indicating that the core has moved to the desired position; and
transmitting, from the controller, a fourth signal to the motor to cease the moving step upon receiving the third signal from the second sensor.
